(19) United States
(12) Patent Application Publication
(10) Pub. No.: US 2024/0307040 A1
(43) Pub. Date: Sep. 19, 2024

(54) VITREOUS SAMPLING DEVICE HAVING VIBRATION-DRIVEN FLUID PRESSURE

(71) Applicant: TWENTY TWENTY THERAPEUTICS LLC, South San Francisco, CA (US)

(72) Inventor: Alexandre R. Tumlinson, South San Francisco, CA (US)

(21) Appl. No.: 18/121,254

(22) Filed: Mar. 14, 2023

(51) Int. Cl. A61B 10/00

(52) U.S. Cl. CPC A61B 10/0045; A61F 9/00745

(57) ABSTRACT

A device for removing vitreous from an eye includes a needle defining a lumen and a port extending to the lumen. The needle is configured for insertion into the vitreous of the eye. A sample container is coupled to the needle and includes a chamber in fluid communication with the lumen and at least at atmospheric pressure. A mechanism is provided for inducing vibration in the needle to draw a sample of the vitreous into the port and to the chamber.

### TECHNICAL FIELD

The subject matter described herein relates to devices, systems, and methods for injection of substances into, and sampling of, aqueous and vitreous humors of the eye. The disclosed intravitreal injection and sampling device has particular but not exclusive utility for diagnosis and treatment of ophthalmic disorders in humans.

### BACKGROUND

Vitreous humor is a colorless, gelatinous fluid within an eye or eyeball of humans or other vertebrates composed of approximately 98-99% water with trace amounts of hyaluronic acid, glucose, anions, cations, ions, and a fine network of collagen. Vitreous humor provides support to the surrounding structures of the eye, absorbs mechanical trauma, and provides circulation and regulation of oxygen, metabolites and nutrients. It is produced largely by cells of the ciliary body. Changes in vitreous structure that occur with aging, are important in the pathogenesis of many vitreoretinal diseases.

Intraocular pressure (TOP) quantifies the pressure of the vitreous humor inside the eye. Many individuals suffer from disorders, such as glaucoma, that are associated with chronic heightened IOP. Over time, heightened IOP can cause damage to the optical nerve of the eye, leading to loss of vision.

Presently, treatment of ophthalmic disorders mainly involves periodically administering pharmaceutical agents to the eye. These drugs can be delivered by, for example, intravitreal injection. Intravitreal injection is one of the most common surgical procedures performed in ophthalmology today. A variety of drugs are delivered directly to the clear vitreous gel that supports the globe of the eye. These drugs act directly in the vitreous or in the surrounding retinal tissues over the following months. For example, intravitreal injection is a common route of delivery for vascular endothelial growth factor inhibiting (anti-VEGF) proteins, which are highly potent compounds tolerated at high doses, with intravitreal half-lives about one week. Anti-VEGF biologics and steroids are the most commonly administered drugs by this route. These drugs may be administered on a chronic basis.

One recommended procedure for intravitreal injection includes preparation of an injection needle, topical anesthesia and disinfection of the eye surface, holding the eye open with a lid speculum or other means, optional lateral dislocation of the conjunctiva at the injection site, and insertion of the needle a few mm lateral to the limbus to approximately the full depth of the needle, injecting the drug, withdrawing the needle, and allowing the conjunctiva to cover the injection site. Post injection care typically includes a basic verification of functional vision such as requesting the patient to count the number of fingers shown by the doctor. This functional test verifies that acute IOP increase due to injection has not impacted the optic nerve head in a way that requires immediate relief.

Another important ophthalmic procedure is vitreous sampling. Vitreous sampling may inform various aspects of eye care. Samples of vitreous may be analyzed for cellular content and extracellular structure by histology or immunologic analysis. Histology can, for example, provide a definitive diagnosis for the type of infection causing endophthalmitis.

Identification of the type of immune cells present and the immune mediator proteins expressed may inform the treatment of uveitis. Identification of the amount of VEGF present in the vitreous may give an indication of how likely imminent neovascularization is to occur or how likely it is that VEGF compounds are responsible for an observed case of neovascularization. Non-responders to anti-VEGF treatment remains one of the most troublesome aspects of treating neovascularization in exudative, age-related vascular degeneration (also known as wet AMD) and diabetic retinopathy.

Two common methods of vitreous sampling—with a cutter or with needle aspiration—appear to be approximately equivalent for the purposes of protein analysis. A state of the art miniature cutting tool may be delivered through a 23-gauge trocar. Needle aspiration may be performed with needles as small as 30-gauge (about half the diameter of 23 gauge). Fine gauge may increase the probability of a dry tap and/or change the properties of the aspirated material by acting as a filter. Small gauge may have an advantage in that traction may not be introduced on the gel matrix because the gel matrix cannot be pulled into the small needle bore. Vitreous samples are typically frozen or otherwise stabilized so that they can be processed in a laboratory outside of the operating room or ophthalmic office setting.

Injection of therapeutic doses of medication into the vitreous or aqueous humor inside the eye can increase TOP by as much as 25 mmHg, which is substantially greater than threshold levels that are considered potentially harmful. Evidence shows that while such TOP increases are transient, they are in fact associated with an iatrogenic glaucoma resulting in measurable loss of nerve fiber layer and visual function over a course of only several treatments in patients with ‘normal’ resting TOP. See Saxena, S., Lai, T. Y., Koizumi, H. et al., “Anterior chamber paracentesis during intravitreal injections in observational trials: effectiveness and safety and effects,” International Journal of Retina and Vitreous, 5, 8 (2019). Therefore, it is sometimes desirable to remove a small volume of humor (whether aqueous, vitreous or both) from the eye before injecting a comparable volume of medication. However, removal of a volume of humor may result in insufficient pressure, which can also be harmful to the eye.

Therefore, in the case of diagnostic sampling of humors, it may be necessary or beneficial to inject a volume of fluid (whether medicated or otherwise) to replace the withdrawn humors. In either case, care must be taken to ensure that the removed and injected volumes are comparable, and in either case, two separate procedures (a sampling procedure and an injection procedure) are typically required. Moreover, it can be beneficial to control the flow of vitreous during sampling to help ensure reliable operation of the sampling device.

### SUMMARY

In one example, a device for removing vitreous from an eye includes a needle defining a lumen and a port extending to the lumen. The needle is configured for insertion into the vitreous of the eye. A sample container is coupled to the needle and includes a chamber in fluid communication with the lumen and at least at atmospheric pressure. A mechanism is provided for inducing vibration in the needle to draw a sample of the vitreous into the port and to the chamber.

In another example, a method for removing vitreous from an eye includes inserting a needle defining a lumen and a port extending to the lumen into the vitreous of the eye. A chamber of a sample container coupled to the needle and in fluid communication with the lumen is maintained at least at atmospheric pressure while inducing ultrasonic vibration in the needle to draw a sample of the vitreous into the port and to the chamber.

In another aspect of the invention, taken alone or in combination with any other aspect, the mechanism is an ultrasonic transducer.

In another aspect of the invention, taken alone or in combination with any other aspect, the ultrasonic transducer is a langevin transducer.

In another aspect of the invention, taken alone or in combination with any other aspect, the ultrasonic transducer operates at a frequency of about 20 kHz to about 50 kHz.

In another aspect of the invention, taken alone or in combination with any other aspect, the ultrasonic transducer energizes piezoelectric crystals to cause a solid acoustic horn to vibrate.

In another aspect of the invention, taken alone or in combination with any other aspect, the ultrasonic transducer energizes piezoelectric crystals to cause a fluid acoustic horn to vibrate.

In another aspect of the invention, taken alone or in combination with any other aspect, a drug delivery syringe is provided for delivering a therapeutic agent through the port.

In another aspect of the invention, taken alone or in combination with any other aspect, the sample container has a rigid connection with the needle for maintaining the needle co-axial with the chamber to allow the syringe to extend through the chamber and into the lumen.

In another aspect of the invention, taken alone or in combination with any other aspect, the sample container has a flexible connection with the needle such that the chamber is laterally displaceable from a centerline of the needle to allow the syringe to bypass the chamber and extend into the lumen.

In another aspect of the invention, taken alone or in combination with any other aspect, structure is provided for sealing a proximal end of the chamber and an actuator for sealing a distal end of the chamber.

In another aspect of the invention, taken alone or in combination with any other aspect, an optical sensor is coupled to the sample container for measuring an optical absorption of the vitreous sample therein to determine a volume of the vitreous sample collected.

In another aspect of the invention, taken alone or in combination with any other aspect, a temperature sensor is coupled to the sample container for measuring a temperature change therein to determine a volume of the vitreous sample collected.

In another aspect of the invention, taken alone or in combination with any other aspect, a pressure sensor is coupled to the sample container for measuring a pressure change therein to determine a volume of the vitreous sample collected.

In another aspect of the invention, taken alone or in combination with any other aspect, a distal end of the needle has a sharpened tip configured for insertion into or through at least one of a sclera, a pars plana or a vitreous body of the eye.

In another aspect of the invention, taken alone or in combination with any other aspect, a controller is configured to control the mechanism for stopping and starting vibration of the needle.

In another aspect of the invention, taken alone or in combination with any other aspect, the port, in combination with the vibrating needle, is configured to liquefy the vitreous by disrupting the integrity of the vitreous gel using at least one of shear force or viscous heat.

Other objects and advantages and a fuller understanding of the invention will be had from the following detailed description and the accompanying drawings.

### BRIEF DESCRIPTION OF THE DRAWINGS

FIG. 1 is a schematic illustration of an example combined biological sampling and injection device in accordance with the present invention.

FIG. 2 is a section view of the device of FIG. 1 taken along line 2-2.

FIG. 3A is a schematic illustration of an example pressure modulation system of the device.

FIG. 3B is a section view of the pressure modulation system of FIG. 3A.

FIG. 4 is an enlarged view of vitreous sampling with the device.

FIG. 5A is a schematic illustration of a first example connection between a sample container and a needle of the device.

FIG. 5B is a schematic illustration of a second example connection between a sample container and a needle of the device.

FIG. 6A is a schematic illustration of another example sampling and injection device.

FIG. 6B is an enlarged view of a portion of the device of FIG. 6A.

FIG. 7 is a schematic illustration of an intermediate step between sampling and injection steps.

FIG. 8 is a schematic illustration of an injection step using the device of FIG. 6A.

### DETAILED DESCRIPTION

The subject matter described herein relates to devices, systems, and methods for injection of substances into, and sampling of, aqueous and vitreous humors of the eye. The disclosed intravitreal injection and sampling device has particular but not exclusive utility for diagnosis and treatment of ophthalmic disorders in humans.

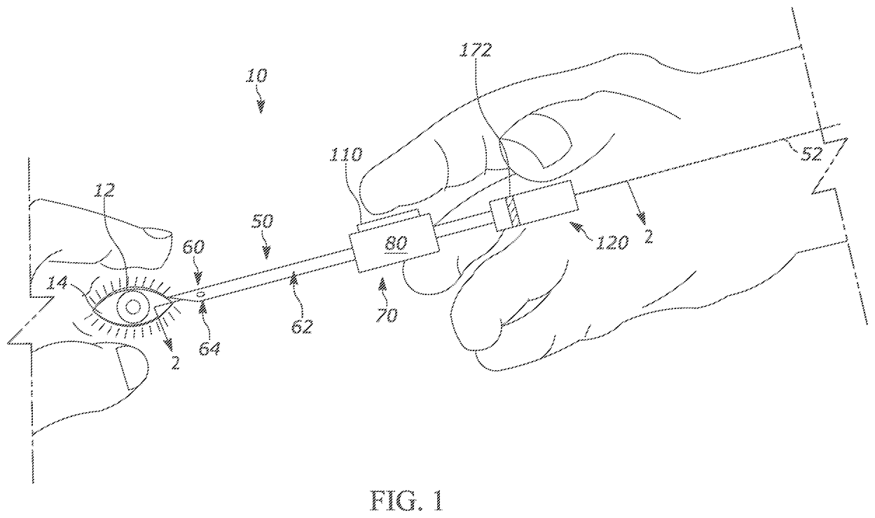

FIG. 1 illustrates an example combined biological sampling and injection assembly or device 10 in accordance with the present invention. The device 10 is operable by a user, e.g., a physician, to obtain a sample of vitreous humor 14 from an eye 12 of a patient.

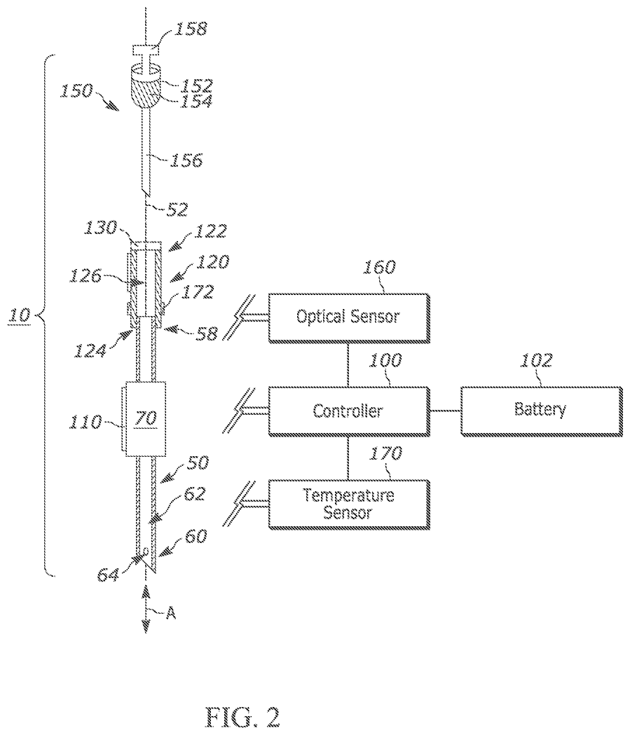

Referring to FIG. 2, the device 10 includes a tubular eye penetration member or needle 50 extending longitudinally along a centerline 52 from a proximal end 58 to a distal end 60. Each end 58, 60 can have a pointed, angled configuration or a blunt/rounded configuration. A lumen 62 extends between the ends 58, 60 along the length of the needle 50. At least one port 64 extends radially from the periphery of the needle 50 radially inward to the lumen 62. It will be appreciated, however, that the port 64 could alternatively or additionally extend longitudinally through the distal end 60 of the needle 50. In other words, the port 64 can be coextensive with the lumen 62 (not shown).

A pressure modulation system or mechanism 70 is provided for modulating the pressure of any fluid delivered to or provided within the lumen 62 and/or modulating movement of the needle 50. In one example, the transducer 70 constitutes a hollow vibration source, such as an ultrasonic transducer. In particular, the ultrasonic transducer can constitute a langevin transducer.

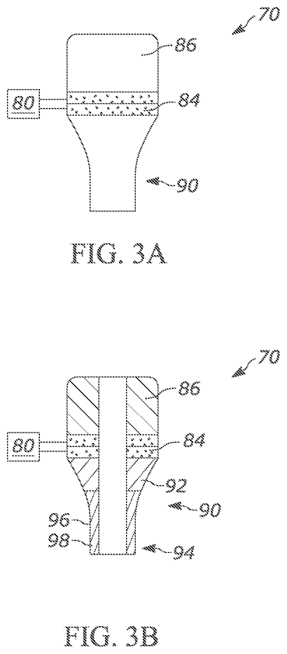

FIGS. 3A-3B illustrate the transducer 70 in more detail. The transducer 70 can be configured to drive an axial vibration of the needle 50 such that the needle tip displacement (stroke length) is on the order of about 5-100 μm at a frequency of about 20 kHz to about 50 kHz. With this in mind, the transducer 70 is coupled to piezoelectric crystals 84 for energizing the same upon activation of an actuator 110 provided along the device 10 (see FIG. 1). A back mass 86 cooperates with the energized piezoelectric crystals 84 to vibrate an acoustic horn 90 connected to the needle 50. The horn 90 can be solid and/or fluid in construction.

In one instance shown in FIG. 3B, the horn 90 includes a solid portion 92 and a liquid portion 94. Both portions 92, 94 in this construction are cylindrical/hollow to accommodate the needle 50. The solid portion 92 is secured/connected to the back mass 86. The liquid portion 94 extends distally away from the solid portion 92 and towards the port(s) 64. The liquid portion 94 can include a tube 96 and a liquid 94, e.g., an aqueous liquid, disposed therein. The longitudinal cross-section of the horn 90 decreases in a direction extending from the back mass 86 towards the distal end 60 of the needle 50.

Returning to FIG. 2, the transducer 70 and actuator 110 are powered by a battery 102 coupled to the needle 50. The actuator 110 is also connected to a controller 100 such that controller controls activation/deactivation of the transducer 70 in response to user interaction with the actuator. In one example, pressing the actuator 110 causes the transducer 70 to apply ultrasonic pressure to the needle 50 such that the entire needle 50 axially vibrates in the manner indicated generally at A.

The device 10 further includes a sample container 120 for connection to the needle 50 to receive a sample of vitreous humor 14 from the eye 12. The sample container 120 is a hollow tube extending from a proximal end 122 to a distal end 124 and defining a central passage or chamber 126. The sample container 120 can be formed from a polymer and be solid, opaque or transparent. In one instance, the needle 50 and sample container 120 are rigidly connected to one another and aligned along the centerline 52.

Structure (illustrated schematically at 130) is provided at or over the proximal end 122 for sealing the proximal end of the chamber 126 in a desired manner. A sealing actuator 172, e.g., a pinching member, is provided around the distal end 124 for sealing the same. In one example, the chamber 126 is actively or passively sealed by the elements 130, 172 to be at or above atmospheric pressure. To this end, the structure 130 can be formed as a hydrophobic barrier or septum that enables the free exchange of gas between the atmosphere and the chamber 126 but prevents liquid from exiting the chamber. Alternatively or additionally, the structure 130 can include a check valve or other conventional sealing structure.

It will be appreciated that the sample container 120 itself can also be configured to help define a chamber 126 at or above atmospheric pressure. For instance, the sample container 120 can be configured to passively expand as the sample of vitreous humor 14 fills the chamber 126. Alternatively or additionally, the sample container 120 can be sealed with an initial pressure of the chamber 126 at or above atmospheric pressure with the chamber pressure increasing as the volume of vitreous humor 14 therein increases.

The device 10 can further include a drug delivery system or syringe 150 for delivering medication to the eye 12 following removal of vitreous humor 14. The drug syringe 150 includes a hollow container 152 for holding a drug 154 of known medication, therapeutic agent, etc. The drug 154 can be in a vial, in liquid form, etc. A needle 156 extends into the container 152 and is in fluid communication with the drug 154. A plunger 158 cooperates with the container 152 to selectively expel the drug 154 out of the container 152 and through the needle 156 in a known manner.

When the device 10 is assembled, the sample container 120 is slid over the proximal end 58 of the needle 50 to establish fluid communication between the lumen 62 and the chamber 126. This places the chamber 126 axially between the proximal end 58 of the needle 50 and the sealing structure 130.

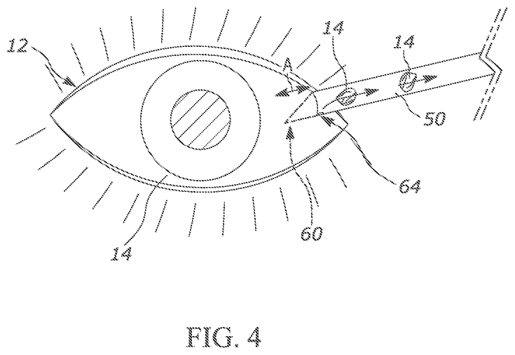

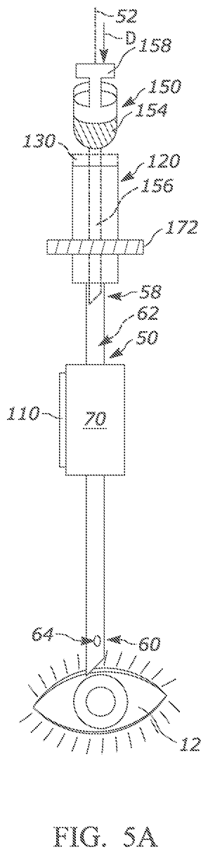

In operation and referring further to FIG. 4, the distal end 60 of the needle 50 is positioned adjacent the eye 12 and inserted therein. Once the distal end 60 is positioned within the vitreous humor 14, the transducer 70 is activated by the user (via the actuator) or automatically activated (in response to sensor readings). In either case, forces are imparted on the vitreous humor 14, including shear and viscous heating, that act to disrupt the integrity of the vitreous humor. More specifically, the transducer 70—by axially vibrating the distal end 60 of the needle 50 in the manner A—breaks apart and liquefies the vitreous humor 14 sufficient to draw portions of the vitreous humor into the radial port(s) 64. The vitreous humor 14 passes radially inward through the port(s) 64 and into the lumen 62 of the needle 50, ultimately passing through the needle, through the passage 99 in the transducer 70, and into the chamber 126.

Advantageously, maintaining a neutral or positive pressure within the chamber 126—as opposed to a vacuum—can help slow down the vitreous humor 14 sampling process. This can help to prevent clogging of the port(s) 64 and/or lumen 62. That said, the vibrating needle 50, in combination with the neutral/positive pressure sample container 152, results in a steady but measured motive force on the vitreous humor 14 drawn into the port(s) 64, through the lumen 62, and ultimately into the chamber 126.

An optical sensor 160 can be positioned on the container 120 and has a probe wavelength transmissive to the chamber 126 walls but opaque to the entering vitreous humor 14 sample. The optical sensor 160 measures the volume of the sample received by the chamber 126 via optical absorption and sends signals indicative thereof to the controller 100. Alternatively or additionally, a temperature sensor 170 can be connected to the controller 100 and used to monitor the level of vitreous sample within the chamber 126 by measuring temperature changes in the chamber. Alternatively or additionally, a pressure sensor (not shown) can be connected to the controller 100 and used to monitor the level of vitreous sample within the chamber 126 by measuring pressure changes in the chamber.

Once the chamber 126 is full, the system times out or the user aborts the sampling procedure, the controller 150 can deactivate the transducer 70 and thereby cease vibration of the needle 50. Regardless, the sealing actuator 172 located adjacent the distal end 124 of the sampling container 120 is then actuated to pinch or close off the sampling container and thereby retain/seal the sample of vitreous humor 14 within the chamber 126.

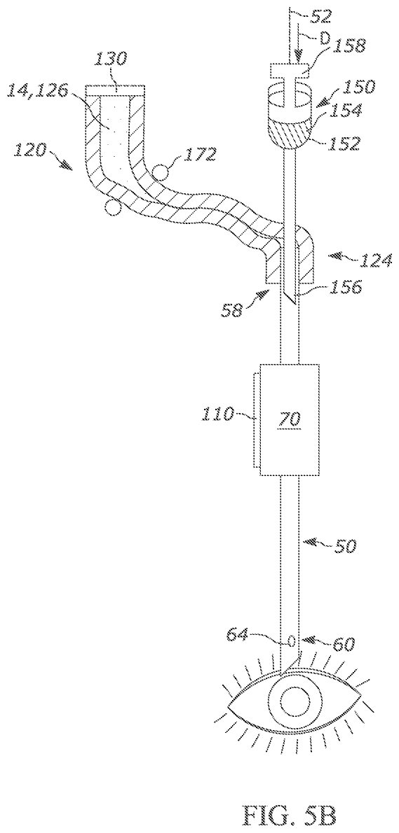

Referring to FIG. 5A, while the needle 50 remains within the eye 12, the syringe 150 is axially aligned with the needle 50. The needle 156 is advanced in the direction D until it pierces and passes through both the sealing structure 130 and the sealing actuator 172 and enters the lumen 62. In other words, the needle 156 passes through the chamber 126 with the sample of vitreous humor 14 therein. This places the needle 156 in fluid communication with the lumen 62 and, thus, in fluid communication with the radial port(s) 64. That said, thereafter depressing the plunger 158 in the direction D pushes the drug 154 out of the container 152, through the needle 156, into the lumen 62 of the needle 50, and out of the device 10 through the radial port(s) 64 to be fully injected into the eye 12 where the vitreous humor 14 sample was removed. After the drug 154 is delivered, the entire device 10 can be retracted out of the eye 12 by pulling the same in the direction opposite the direction D.

In another, modified example device 10 shown in FIG. 5B, the sample container 120 is formed from a resiliently deflectable material, such as rubber. This configuration allows the sample container 120 to deflect relative to the centerline 52 without compromising the integrity of the chamber 126. That said, once the vitreous humor 14 sample is taken from the eye 12, the sample container 120 can be laterally deflected so as to be offset from or transverse to the centerline 52. The sealing actuator 172 is then used to pinch or otherwise seal off the distal end of the chamber 126. The deflected distal end 124 of the sample container 120 acts to cover and seal and proximal end 58 of the needle 50.

The needle 156 is then advanced in the direction D until it pierces and passes through the deflected, distal end 124 of the sample container 120 and enters the lumen 62. This places the needle 156 in fluid communication with the lumen 62 and, thus, in fluid communication with the radial port(s) 64. Moreover, laterally displacing the sample container 120 removes the chamber 126 and vitreous humor 14 therein from the flow path between the syringe 150 and the lumen 62.

That said, thereafter depressing the plunger 158 in the direction D pushes the drug 154 out of the container 152, through the needle 156, into the lumen 62 of the needle 50, and out of the device 10 through the radial port(s) 64 to be fully injected into the eye 12 where the vitreous humor 14 sample was removed. After the drug 154 is delivered, the entire device 10 can be retracted out of the eye 12 by pulling the same in the direction opposite the direction D. The sealing structure 130 and sealing actuator 172 cooperate to maintain the vitreous humor 14 sample within the chamber 126 during the drug injection phase of the procedure.

It is clear from the above that the sample container 120 can be configured to have a relatively more rigid construction (FIG. 2) such that the sample container remains longitudinally aligned with both the needle 50 and the syringe 150 during the sampling/drug delivery or a relatively more flexible construction (FIG. 5A) such that the sample container is laterally displaced from the aligned needle 50 and syringe 150.

In either case, the device of the present invention is advantageous in that the combination of ultrasonic vibration and a neutral/positive pressure in the sample container chamber help to retard the flow of vitreous humor into the device. Furthermore, eliminating the need for a vacuum sealed sample container simplifies the device by, for example, obviating the need to ensure a vacuum is maintained over time for storage, transport, etc. Further to this point, removing the need for a vacuum also loosens the design requirements of the sample container related to gas permeability and/or rigidity.

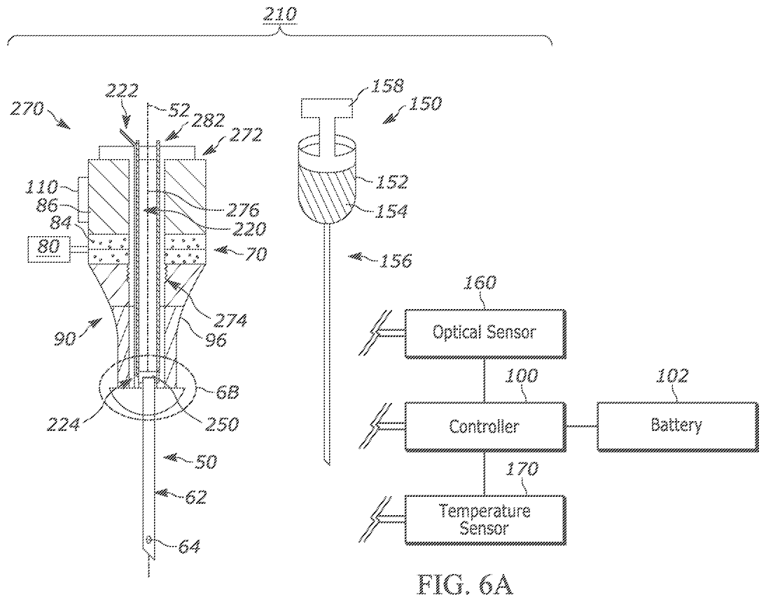

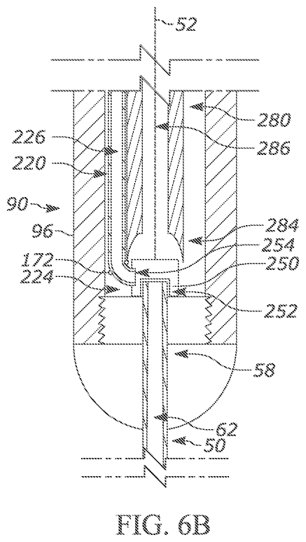

FIGS. 6A-6B illustrate another example device 210 in accordance with the present invention. Features in FIGS. 6A-6B that are similar to those in FIG. 2 carry the same reference numbers.

In FIG. 6A, the sample container 220 is configured as an elongated tube extending substantially the entire length of the transducer 70. More specifically, the sample container 220 extends generally parallel to the centerline 52 and from a proximal end 222 positioned proximal to the transducer 70 and backmass 86 to a distal end 224 positioned within the horn 90. The sample container 220 defines a chamber 226 extending substantially the entire length thereof.

In one example, a hollow cap 250 is provided within the horn 90 adjacent the proximal end 58 of the needle 50. The cap 250 includes a first receiving portion 252 for connecting to the proximal end 58 and a second receiving portion 254 for connecting to the distal end 224 of the sample container 220. Consequently, the cap 250 fluidly connects the chamber 226 with the lumen 62.

A compression bolt 270 extends along the centerline and within the transducer 70. The bolt 270 extends from a proximal end 272 positioned outside the backmass 86 to a distal end 274 secured to the horn 90, e.g., via threading or the like. A central passage 276 extends the entire length of the bolt 270. A guide tube 280 extends within the compression bolt 270 from a proximal end 282 extending out of the proximal end 272 to a pointed distal end 284 positioned within the tube 96 of the horn 90. The second end 284 is aligned with the distal end 224 of the sample container 220 and positioned radially outward of the cap 250. A central passage 286 extends the entire length of the guide tube 280 and terminates at the cap 250.

When the device 210 is assembled, the guide tube 280 is initially spaced from the distal end 224 of the sample container 220. The transducer 70 encircles and extends generally parallel to the exterior of the guide tube 280. The sample container 220 extends in a radial gap between the bolt 270 and the guide tube 280.

The distal end 60 of the needle 50 is advanced into the eye 12 and the user actuates the actuator 110 to thereby activate the ultrasonic transducer 70 to induce reciprocating, axial movement A of the distal end 60 along the centerline 52. This draws the vitreous humor 14 sample through the port(s) 64, into the lumen 62, and upwards (as shown) through the cap 250, and into the chamber 226. This flow can be controlled by providing the chamber 226 at or above atmospheric pressure.

The sample of vitreous humor 14 can be collected until the sample is visible within the proximal end 222 of the sample container 220. In other words, the sample can be collected until it is visible by the user proximal to the backmass 86. Alternatively or additionally, either of the sensors 160, 170 can be implemented to allow the controller 100 to track the volume of vitreous humor 14 collected.

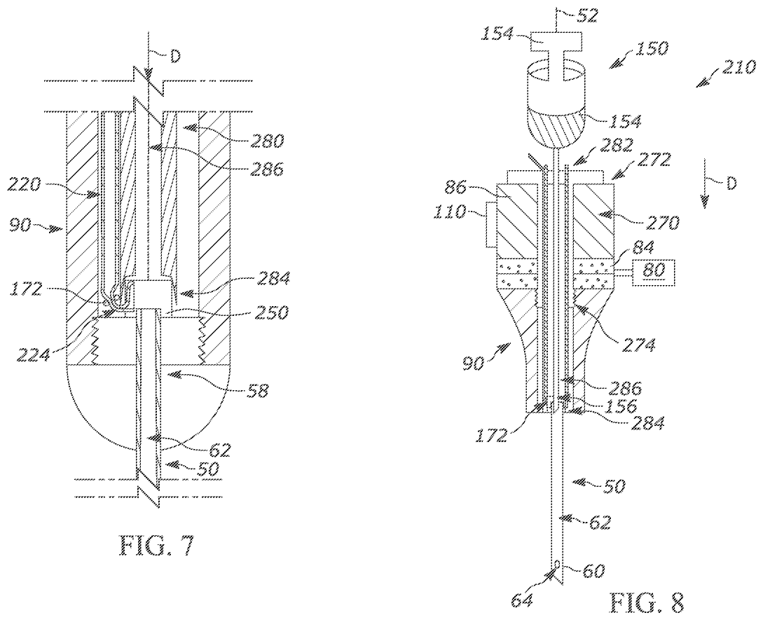

In any case, and referring to FIG. 7, once an adequate sample size is taken, the user can urge the guide tube 280 in the direction D relative to the transducer 70 and the needle 50. When this occurs, the pointed distal end 284 of the guide tube 280 pinches and seals the distal end 224 of the sample container 220. This fluidly separates the chamber 226 from the interior of the cap 250 and thereby fluidly separates the chamber 226 from the lumen 62.

It will be appreciated that movement of the guide tube 280 in the manner D can completely sever the distal end 224 of the sample container 220 from the distalmost portion of the sample container coupled to the cap 250. The controller 100 can maintain the ultrasonic transducer 70 in the activated state while this severing occurs, which allows the ultrasonic energy to help seal the distal end 224 and thereby seal the sample within the chamber 226. In particular, the ultrasonic energy can generate heat to weld the polymer layers of the distal end 224 of the sample container 220 shut. This sealing can be advantageous in allowing the sample container 220—with the vitreous humor 14 sample sealed therein—to be removed from the device 210 prior to the drug injection phase of the procedure.

Turning to FIG. 8, the drug delivery syringe 150 for this device 220 in this configuration has a significantly longer needle 156 than the syringe used with the device 10. That said, once the sample container 220 is sealed, the syringe 150 is aligned with the guide tube 280 and the needle 156 advanced in the direction D through the passage 286. The needle 156 eventually passes through the cap 250 and enters the lumen 62 of the needle 50. This places the needle 156 in fluid communication with the lumen 62 and, thus, in fluid communication with the radial port(s) 64. Moreover, laterally displacing the sample container 120 (by pinching/separating it from cap 250) removes the chamber 126 and vitreous humor 14 therein from the flow path between the syringe 150 and the lumen 62.

That said, thereafter depressing the plunger 158 in the direction D pushes the drug 154 out of the container 152, through the needle 156, into the lumen 62 of the needle 50, and out of the device 210 through the radial port(s) 64 to be fully injected into the eye 12 where the vitreous humor 14 sample was removed. After the drug 154 is delivered, the entire device 210 can be retracted out of the eye 12 by pulling the same in the direction opposite the direction D.

It will be appreciated that additional, alternative configurations for the modulation mechanism can be implemented into the assemblies of the present invention. To this end, the modulation mechanisms can be mechanical-based and formed as a drill bit inside the needle or rely on oscillating guillotine blades driven by a variety of motor types.

The modulation mechanisms can be non-mechanical in nature. For instance, an energy delivery device can be provided on the distal end of the needle for helping to denature the vitreous gel in the eye. In one example, the energy delivery device includes one or more electrodes arranged circumferentially about the distal end of the needle and selectively energized by the controller. Furthermore, the electrodes can be arranged along the interior and/or exterior of the distal end of the needle.

Alternatively or additionally, one or more optical fibers can be provided on the distal end of the needle (along the interior and/or exterior thereof) for delivering a high energy pulse at a wavelength highly absorbed by water to locally disrupt the vitreous gel. The optical fibers can also be connected to and controlled by the controller.

Moreover, targeted Proteases such as collagenase, hyaluronidase, or others may be used to break down the specific proteins of the vitreous gel. These may be injected directly before the sampling or hours or weeks before sampling. Traditional chemicals such as strong acids or bases that are not particularly targeted may be injected alone or in combination with proteases to speed the chemical action to break down the structural proteins of vitreous.

The combined biological sampling and injection assemblies described herein are advantageous in that they provide for a small gauge, e.g., 30G or smaller, instrument that can directly penetrate the sclera, does not require an external fluid source for operation, and can be operated in a cordless manner.

What have been described above are examples of the present invention. It is, of course, not possible to describe every conceivable combination of components or methodologies for purposes of describing the present invention, but one of ordinary skill in the art will recognize that many further combinations and permutations of the present invention are possible. Accordingly, the present invention is intended to embrace all such alterations, modifications and variations that fall within the spirit and scope of the appended claims.

What is claimed is:

1. A device for removing vitreous from an eye, comprising:
a needle defining a lumen and a port extending to the lumen, the needle being configured for insertion into the vitreous of the eye;
a sample container coupled to the needle and having a chamber in fluid communication with the lumen and being at least at atmospheric pressure; and
a mechanism for inducing vibration in the needle to draw a sample of the vitreous into the port and to the chamber.

2. The device recited in claim 1, wherein mechanism comprises an ultrasonic transducer.

3. The device recited in claim 2, wherein the ultrasonic transducer is a langevin transducer.

4. The device recited in claim 2, wherein the ultrasonic transducer operates at a frequency of about 20 kHz to about 50 kHz.

5. The device recited in claim 2, wherein the ultrasonic transducer energizes piezoelectric crystals to cause a solid acoustic horn to vibrate.

6. The device recited in claim 2, wherein the ultrasonic transducer energizes piezoelectric crystals to cause a fluid acoustic horn to vibrate.

7. The device recited in claim 1, further comprising a drug delivery syringe for delivering a therapeutic agent through the port.

8. The device recited in claim 7, wherein the sample container has a rigid connection with the needle for maintaining the needle co-axial with the chamber to allow the syringe to extend through the chamber and into the lumen.

9. The device recited in claim 7, wherein the sample container has a flexible connection with the needle such that the chamber is laterally displaceable from a centerline of the needle to allow the syringe to bypass the chamber and extend into the lumen.

10. The device recited in claim 1, further comprising structure for sealing a proximal end of the chamber and an actuator for sealing a distal end of the chamber.

11. The device recited in claim 1, further comprising an optical sensor coupled to the sample container for measuring an optical absorption of the vitreous sample therein to determine a volume of the vitreous sample collected.

12. The device recited in claim 1, further comprising a temperature sensor coupled to the sample container for measuring a temperature change therein to determine a volume of the vitreous sample collected.

13. The device recited in claim 1, further comprising a pressure sensor coupled to the sample container for measuring a pressure change therein to determine a volume of the vitreous sample collected.

14. The device recited in claim 1, wherein a distal end of the needle comprises a sharpened tip configured for insertion into or through at least one of a sclera, a pars plana or a vitreous body of the eye.

15. The device recited in claim 1, further comprising a controller configured to control the mechanism for stopping and starting vibration of the needle.

16. The device recited in claim 1, wherein the port, in combination with the vibrating needle, is configured to liquefy the vitreous by disrupting the integrity of the vitreous gel using at least one of shear force or viscous heat.

17. A method for removing vitreous from an eye, comprising:
inserting a needle defining a lumen and a port extending to the lumen into the vitreous of the eye; and
maintaining a chamber of a sample container coupled to the needle and in fluid communication with the lumen at least at atmospheric pressure while inducing ultrasonic vibration in the needle to draw a sample of the vitreous into the port and to the chamber.

18. The method recited in claim 17 further comprising placing a drug delivery syringe in fluid communication with the lumen for delivering a therapeutic agent through the port.

19. The method recited in claim 17, wherein the sample container has a rigid connection with the needle for maintaining the needle co-axial with the chamber to allow the syringe to extend through the chamber and into the lumen.

20. The method recited in claim 17, wherein the sample container has a flexible connection with the needle such that the chamber is laterally displaceable from a centerline of the needle to allow the syringe to bypass the chamber and extend into the lumen.
